(19) United States
(12) Patent Application Publication
(10) Pub. No.: US 2022/0110709 A1
(43) Pub. Date: Apr. 14, 2022

(54) TROCAR ASSEMBLY FOR ADAPTER ASSEMBLIES WITH HARD-STOP

(71) Applicant: Covidien LP, Mansfield, MA (US)

(72) Inventors: David E. VALENTINE, Hamden, CT (US); Charles R. KOLLAR, Washington, DC (US); Alexander J. HART, Tolland, CT (US); Joseph EISINGER, Northford, CT (US); James P. DELBO, North Haven, CT (US); Joseph D. MALLOZZI, Brewster, NY (US); Keith I. ALLSOP, Bolton, CT (US); Ramiro D. CABRERA, Cheshire, CT (US); Stephen R. PAUL, Burlington, CT (US); Anthony SGROI, Southbury, CT (US); Stephen R. CASEY, Wallingford, CT (US); Haley STRASSNER, Hamden, CT (US); Patrick MOZDZIERZ, Glastonbury, CT (US)

(21) Appl. No.: 17/499,315

(22) Filed: Oct. 12, 2021

Related U.S. Application Data
(60) Provisional application No. 63/089,707, filed on Oct. 9, 2020.

(51) Int. Cl. A61B 90/00; A61B 17/115

(52) U.S. Cl. CPC A61B 90/03; A61B 2017/00473; A61B 17/1155

(57) ABSTRACT

A trocar assembly for releasable engagement with an adapter assembly of a surgical stapling instrument includes housing, a trocar member slidably supported within the housing between a retracted position and an advanced position, and a chive member rotatably supported within the housing and in operable engagement with the trocar member to cause longitudinal movement of the trocar member relative to the housing between an advanced position and a retracted position. The housing includes a tubular body having a distal portion and an end cap assembly secured to the distal portion. The trocar member includes a proximal portion and at least one tab disposed on the proximal portion. The at least one tab is configured to engage the end cap assembly when the trocar member is in the advanced position to prevent further advancement of the trocar member.

### CROSS-REFERENCE TO RELATED APPLICATION

This application claims the benefit of and prior to U.S. Provisional Patent Application No. 63/089,707, filed on Oct. 9, 2020, the content of which is incorporated herein by reference in its entirety.

### FIELD

The disclosure relates to adapter assemblies for circular surgical stapling instruments. More particularly, the disclosure relates to adapter assemblies having a trocar assembly with a hard-stop.

### BACKGROUND

Surgical instruments for applying staples, clips, or other fasteners to tissue are well known. Typically, endoscopic stapling instruments include an actuation unit, i.e., a handle assembly for actuating the instrument, an elongate shaft releasably securable to the actuation unit for accessing a body cavity, i.e., an adapter assembly, and a tool assembly, i.e., a reload assembly and an anvil assembly, disposed at a distal end of the elongate shaft.

Adapter assemblies used with a circular stapling tool assembly include a trocar assembly having a trocar member that is advanceable and retractable relative to a reload assembly to position an anvil assembly relative to the reload assembly. Advancement of the trocar member in excess of its operable range may result in separation of the trocar member from the stapling assembly. Separation of the trocar member from the stapling assembly may unnecessary complicate the surgical procedure.

### SUMMARY

A trocar assembly for releasable engagement with an adapter assembly of a surgical stapling instrument is provided. The trocar assembly includes a housing, a trocar member slidably supported within the housing between a retracted position and an advanced position, and a. drive member rotatably supported within the housing and in operable engagement with the trocar member. The housing includes a tubular body having a distal portion and an end cap assembly secured to the distal portion. The trocar member includes a proximal portion and at least one tab disposed on the proximal portion. The drive member being configured to cause longitudinal movement of the trocar member relative to the housing between the advanced position and the retracted position. The at least one tab is configured to engage the end cap assembly when the trocar member is in the advanced position to prevent further advancement of the trocar member.

In certain aspects of the disclosure, the end cap assembly includes a cap member and an alignment spacer. The trocar member may engage the alignment spacer when the trocar member is in the advanced position. The housing and the trocar member may be rotationally fixed relative to each other. Rotation of the drive member in a first direction may cause advancement of the trocar member and rotation of the drive member in a second direction may cause retraction of the trocar member.

In aspects of the disclosure, the trocar member includes at least one flattened outer surface and the end cap assembly includes at least one flattened inner surface. Cooperation between the at least on inner surface and at least one outer surface may maintain the trocar member rotationally fixed relative to the trocar housing. The housing may define at least one slot for facilitating attachment of the trocar assembly within an adapter assembly. A distal end of the trocar member may include a trocar spike for operable engagement with an anvil assembly. The distal end of the trocar member may include a tapered end.

### BRIEF DESCRIPTION OF THE DRAWINGS

The accompanying drawings, which are incorporated in and constitute a part of this specification, illustrate aspects of the disclosure and, together with a general description of the disclosure given above, and the detailed description of the aspects given below, serve to explain the principles of the disclosure, wherein:

FIG. 1 is a perspective view of a surgical stapling instrument including a trocar assembly according to aspects of the disclosure;

FIG. 2 is a perspective view of an adapter assembly of the surgical stapling instrument shown in FIG. I with a trocar member of the trocar assembly in a partially extended position;

FIG. 3 is a perspective view of the trocar assembly of the surgical stapling instrument shown in FIG. 1;

FIG. 4 is a perspective view of the trocar member of the trocar assembly shown in FIG. 3:

FIG. 5 is an enlarged area of detail shown in FIG. 4;

FIG. 6 is a cross-sectional top view taken along line 6-6 shown in FIG. 2; and

FIG. 7 is an enlarged view of the indicated area of detail shown in FIG. 6.

### DETAILED DESCRIPTION

Aspects of the disclosed trocar assembly will now be described in detail with reference to the drawings in which like reference numerals designate identical or corresponding elements in each of the several views. As is common in the art, the term “proximal” refers to that part or component closer to the user or operator, i,e. surgeon or clinician, while the term “distal” refers to that part or component farther away from the user.

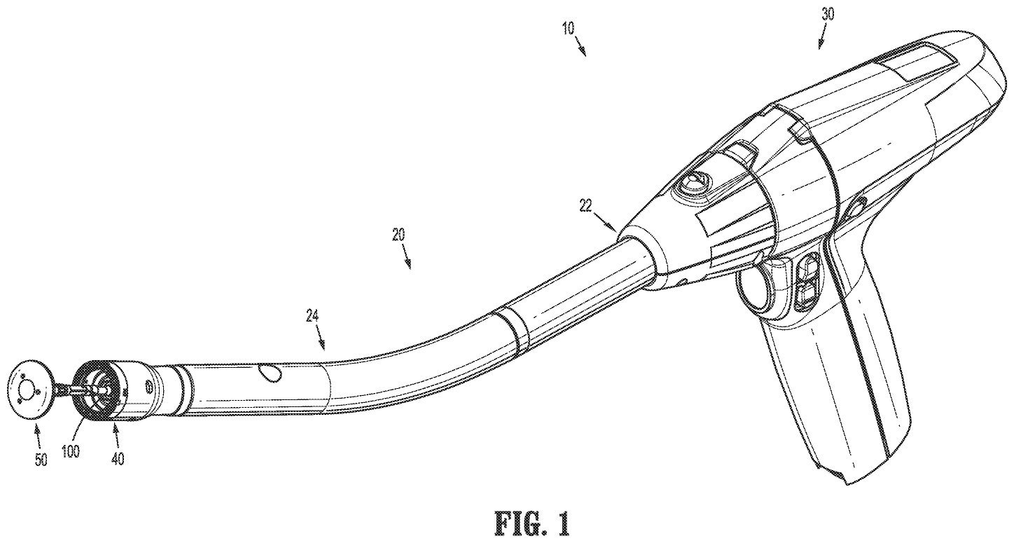

FIG. 1 illustrates an adapter assembly suitable for use with a removable trocar assembly according to aspects of the disclosure, shown generally as adapter assembly 20, is a component of a surgical stapling instrument 10. The surgical stapling instrument 10 further includes a powered handle assembly 30, a loading unit 40, and an anvil assembly 50. Although shown and described with reference to surgical stapling instrument 10, the aspects of the present disclosure may be modified for use with surgical stapling instruments having alternative configurations. For a detailed description of exemplary powered surgical stapling instruments, please refer to commonly owned U.S. Pat. Nos. 9,023,014 and 9,055,943.

The adapter assembly 20 of the surgical stapling instrument 10 will only be described to the extent necessary to fully disclose the aspects of the present disclosure. For a detailed description of exemplary adapter assemblies, please refer to commonly owned U.S. Pat. Nos. 10,226,254 and 10,111,684 (“the '684 patent”).

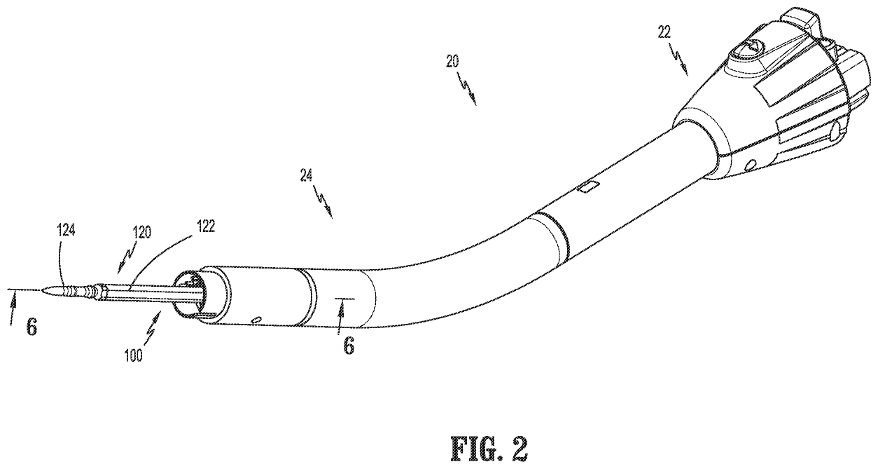

FIG. 2 illustrates the adapter assembly 20 including a proximal portion 22 configured for operable connection to the handle assembly 30 (FIG. 1) and a distal portion 24 configured for operable connection to the loading unit 40 (FIG. 1). Although shown and described as forming an integral unit, it is envisioned that the proximal and distal portions 22, 24 may be formed as separate units that are releasably securable to one another.

A trocar assembly 100 extends distally from the distal portion 24 of the adapter assembly 20 of the surgical stapling instrument 10. The trocar assembly 100 is releasably secured within the distal portion 24 of the adapter assembly 20 by a locking mechanism (not shown). For a detailed description of an exemplary locking mechanism for securing the trocar assembly 100 within the distal portion 24 of the adapter assembly 20, please refer to the '684 patent.

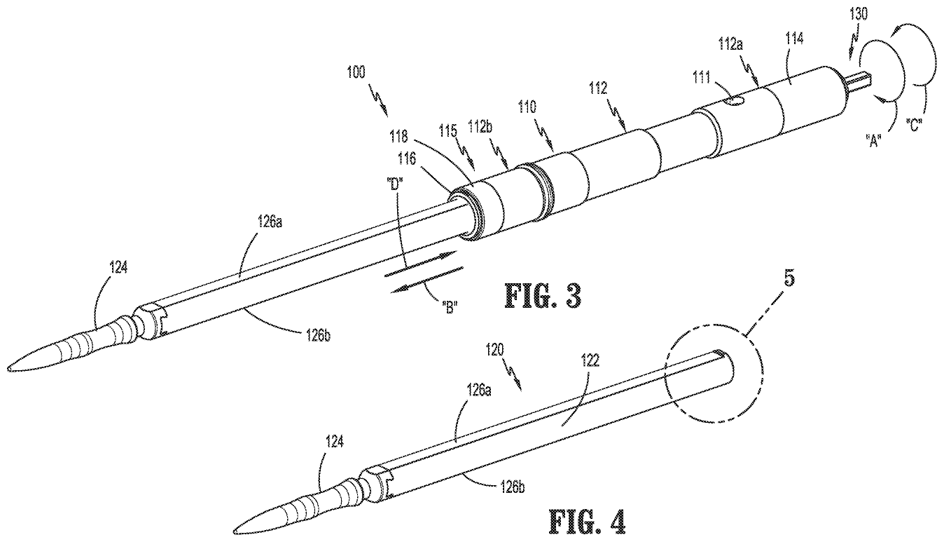

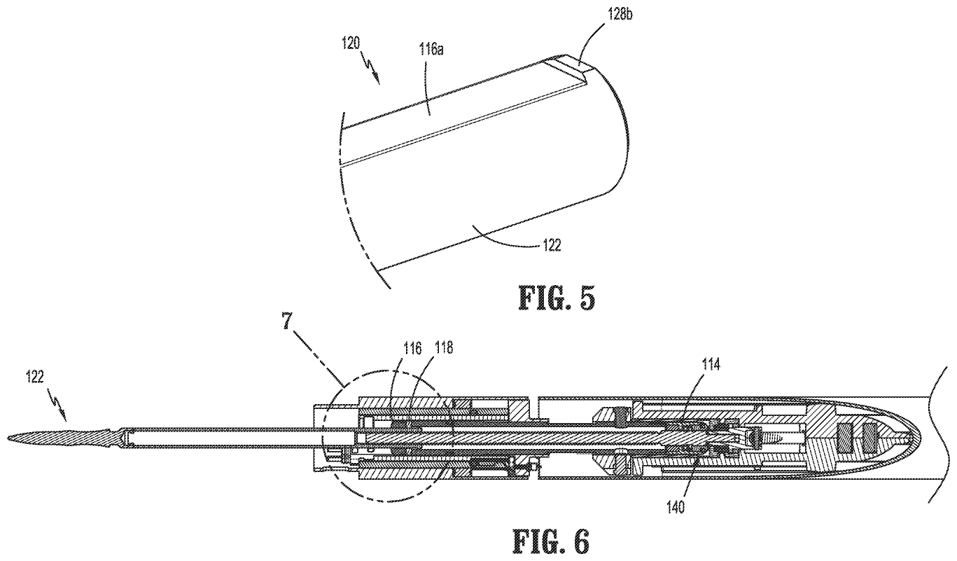

FIG. 3 illustrates the trocar assembly 100 of the adapter assembly 10( )(FIG. 2) of the surgical stapling instrument 10 (FIG. 1) including a trocar housing 110, a trocar member 120 extendable from within the trocar housing 110, and a drive member 130 rotatably supported by the housing 110, and in operable engagement with the trocar member 120. Rotation of the drive member 130 of the trocar assembly 100 in a first rotational direction, i.e., clockwise, as indicated by arrow “A”, causes longitudinal movement of the trocar member 120 in a first longitudinal direction, i.e., advancement, as indicated by arrow “B”, Conversely, rotation of the drive member 130 of the trocar assembly 100 in a second rotational direction, i.e., counter-clockwise, as indicated by arrow “C”, causes longitudinal movement of the trocar member 120 in a second longitudinal direction, i.e., retraction, as indicated by arrow “D”.

The trocar housing 110 of the trocar assembly 100 includes a substantially tubular body 112 having proximal and distal portions 112a, 112b. In certain aspects of the disclosure, and as shown, the tubular body 112 defines a pair of opening 111.1 (only one shown) to facilitate releasable attachment of the trocar assembly 100 within the distal portion 24 of the adapter assembly 20 of the surgical stapling instrument 10. Alternatively, the tubular body 112 of the housing 110 may include tabs, slots and tabs, threading, or other suitable feature for releasable attachment of the trocar assembly 100 to the adapter assembly 20. As noted above, an exemplary locking mechanism for securing the trocar assembly 100 within the distal portion 24 of the adapter assembly 20 is disclosed in the '684 patent.

The trocar assembly 100 includes a proximal end cap 114 disposed on the proximal portion 112a of the tubular body 112. The proximal end cap 114 supports the drive member 130. More particularly, the proximal end cap 114 supports a bearing assembly 140 (FIG. 6) that rotationally supports the drive member 130. As noted above, rotation of the drive member 130 cause longitudinal movement of the trocar member 120 relative to the trocar housing 110. The proximal end cap 114 is secured to the proximal portion 112a of the tubular body 112 of the housing 110 in any suitable manner, including with, for example, adhesive, welding, friction fit, or mechanical interface, e.g., threads or bayonet coupling.

The trocar assembly 100 also includes a distal end cap assembly 115 disposed on the distal portion 112b of the tubular body 112, The distal end cap assembly of the trocar assembly 100 supports the trocar member 120 of the trocar assembly 100 as the trocar member 120 moves longitudinally relative to the trocar housing 110. More particularly, the distal end cap assembly 115 includes an end cap member 116 supporting an alignment spacer 118, and both the distal end cap member 116 and the alignment spacer 118 support the trocar member 120. As will be described in further detail below, the alignment spacer 118 is configured to engage a tab 124 of the trocar member 120 to prevent the trocar member 120 from extending beyond the trocar housing 110. Although shown as separate components, it is envisioned that the distal end cap 116 and the alignment spacer 118 may be integrally formed.

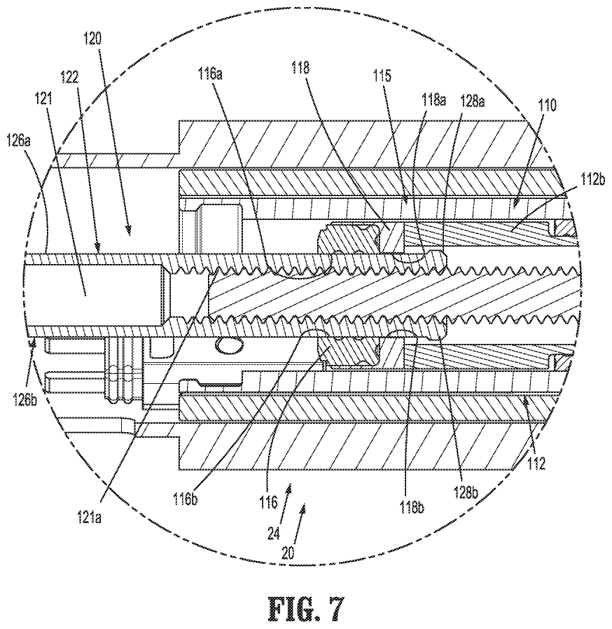

Each of the distal end cap 116 and the alignment spacer 118 of the trocar assembly 100 include first and second flattened inner surfaces 116a (FIG. 7), 118a, 116b, 118b. As will be described below, the first and second flattened inner surfaces 116a, 118a, 116b, 118b of the respective distal end cap 116 and alignment spacer 118 cooperate with the first and second flattened outer surfaces 126a, 126b, respectively, of the trocar member 120 of the trocar assembly 100, to rotationally fix the trocar member 120 relative to the trocar housing 110.

FIG. 4 illustrates the trocar member 120 of the trocar assembly 100 of the adapter assembly 20 (FIG. 2) of the surgical stapling instrument 10 (FIG. 1). The trocar member 120 includes an elongate body 122 and a trocar spike 124 supported on an end of the elongate body 122. Although shown as separate components, it is envisioned that the trocar spike 124 may be integrally formed with the elongate body 122. The trocar spike 124 of the trocar member 120 is configured to penetrate tissue and permit releasable engagement with an anvil assembly, e.g., anvil assembly 50 (FIG. 1). The elongate body 122 of the trocar member 120 includes first and second flattened outer surfaces 126a, 126b. The first and second flattened outer surfaces 126a, 126b of the elongate body 122 align with the first and second flattened inner surfaces 116a (FIG. 7), 118a, 116b, 118b of the respective distal end cap 116 and alignment spacer 118. In this manner, the trocar member 120 is prevented from rotating as the trocar member 120 moves longitudinally relative to the trocar housing 110.

Adjacent the proximal ends of the respective first and second flattened surfaces 126a, 126b (FIG. 7) of the elongate body 122 of the trocar member 120, the elongate body 122 includes first and second tabs 128a, 128b. Although shown including first and second flattened outer surfaces 126a, 126b and corresponding first and second tabs 128a, 128b, it is envisioned that the elongate body 122. may include only a single flattened outer surface, or may include more than two flattened outer surfaces, any of which may include a tab. In certain aspects of the disclosure, the elongate body 122 of the trocar member 120 may include a flange (not shown) extending partially or entirely around a distal portion of the elongate body 122.

As will be detailed below, the first and second tabs 128a, 128b formed on the elongate body 122 of the trocar member 120 act as a hard-stop to prevent the trocar member 120 from separating from the trocar housing 110. The hard-stop also allows software within the powered handle assembly 30 (FIG. 1) of the surgical stapling instrument 10 to calibrate the position of the trocar spike 124 by extending the trocar member 120 More particularly, the trocar member 120 may be extended until the hard-stop is achieved, and without separating the trocar member 120 from the trocar housing 110, thereby providing the software in the powered handle assembly 30 the relative position of the trocar spike 124 relative to the trocar housing 110. In this manner, calibration of the position of the trocar spike 124 may be achieved in the customary manner of retracting the trocar member 120 relative to the trocar housing 110, or by extending the trocar member 120 until achieving the hard-stop.

The trocar member 120 defines a longitudinal channel 121 (FIG. 7) including at least a portion that is threaded, i.e., threaded portion 121a (FIG. 7). The longitudinal channel 121 of the trocar member 120 is configured to receive a threaded portion 134 of the drive member 130, Engagement of the threaded portion 121a of the trocar member 120 with the threaded portion 134 of the drive member 130 cause longitudinal movement of the trocar member 120.

FIGS. 6 and 7 illustrates the adapter assembly 20 of the surgical stapling instrument 10 (FIG. 1) with the trocar member 110 of the trocar assembly 100 in a partially extended position, In this partially extended position, the first and second tabs 128a, 128b on the elongate body 122 of the trocar member 120 are spaced from the alignment spacer 118 disposed on a distal portion 112b of the tubular body 112 of the trocar housing 110.

Further advancement of the trocar member 120 of the trocar assembly 100 causes the first and second tabs 128a, 128b on the elongate body 122 of the trocar member 120 to engage the alignment spacer 118 of the trocar housing 110, thereby providing a hard-stop for the advancement of the trocar member 120. As described above, the hard-stop permits the software to calibrate the position of the trocar spike 124 (FIG. 6) by advancing the trocar member 120. Depending on the initial position of the trocar member 120 relative to the trocar housing 110, extending the trocar member 120 may be preferable to retracting the trocar member 120 when calibrating the position of the trocar spike 124.

Although the illustrative aspects of the disclosure have been described herein with reference to the accompanying drawings, it is to be understood that the disclosure is not limited to those precise aspects, and that various other changes and modifications may be effected therein by one skilled in the art without departing from the scope or spirit of the disclosure.

What is claimed is:

1. A trocar assembly operably engageable with an adapter assembly of a surgical stapling instrument, the trocar assembly comprising:
a housing including a tubular body having a distal portion and an end cap assembly secured to the distal portion;
a trocar member slidably supported within the housing between a retracted position and an advanced position, the trocar member including a proximal portion and at least one tab disposed on the proximal portion; and
a drive member rotatably supported within the housing and in operable engagement with the trocar member to cause longitudinal movement of the trocar member relative to the housing between the advanced position and the retracted position, wherein the at least one tab is configured to engage the end cap assembly when the trocar member is in the advanced position to prevent further advancement of the trocar member.

2. The trocar assembly of claim 1, wherein the end cap assembly includes a cap member and an alignment spacer.

3. The trocar assembly of claim 2, wherein the trocar member engages the alignment spacer when the trocar member is in the advanced position.

4. The trocar assembly of claim 1, wherein the housing and the trocar member rotationally fixed relative to each other.

5. The trocar assembly of claim 4, wherein rotation of the drive member in a first direction causes advancement of the trocar member.

6. The trocar assembly of claim 5, wherein rotation of the drive member in a second direction causes retraction of the trocar member.

7. The trocar assembly of claim 1, wherein the trocar member includes at least one flattened outer surface and the end cap assembly includes at least one flattened inner surface, wherein cooperation between the at least on inner surface and at least one outer surface maintain the trocar member rotationally fixed relative to the trocar housing.

8. The trocar assembly of claim 1, wherein the housing defines at least one slot for facilitating attachment of the trocar assembly within an adapter assembly.

9. The trocar assembly of claim 1, wherein a distal end of the trocar member includes a trocar spike for operable engagement with an anvil assembly.

10. The trocar assembly of claim 9, wherein the distal end of the trocar member includes a tapered end.

11. A surgical stapling instrument comprising:
an adapter assembly; and
a trocar assembly operably engageable with the adapter assembly, the trocar assembly including,
a housing including a tubular body having a distal portion and an end cap assembly secured to the distal portion;
a trocar member slidably supported within the housing between a retracted position and an advanced position, the trocar member including a proximal portion and at least one tab disposed on the proximal portion; and
a drive member rotatably supported within the housing and in operable engagement with the trocar member to cause longitudinal movement of the trocar member relative to the housing between the advanced position and the retracted position, wherein the at least one tab is configured to engage the end cap assembly when the trocar member is in the advanced position to prevent further advancement of the trocar member.

12. The surgical stapling instrument of claim 11, wherein the end cap assembly includes a cap member and an alignment spacer.

13. The surgical stapling instrument of claim 12, wherein the trocar member engages the alignment spacer when the trocar member is in the advanced position.

14. The surgical stapling instrument of claim 11, wherein the housing and the trocar member rotationally fixed relative to each other.

15. The surgical stapling instrument of claim 14, wherein rotation of the drive member in a first direction causes advancement of the trocar member.

16. The surgical stapling instrument of claim 15, wherein rotation of the drive member in a second direction causes retraction of the trocar member.

17. The surgical stapling instrument of claim 11, wherein the trocar member includes at least one flattened outer surface and the end cap assembly includes at least one flattened inner surface, wherein cooperation between the at least on inner surface and at least one outer surface maintain the trocar member rotationally fixed relative to the trocar housing.

18. The surgical stapling instrument of claim 11, wherein the housing defines at least one slot for facilitating attachment of the trocar assembly within the adapter assembly.

19. The surgical stapling instrument of claim 11, further including an anvil assembly, wherein a distal end of the trocar member includes a trocar spike for operable engagement with the anvil assembly.

20. A surgical stapling instrument comprising:
an actuation assembly;
an adapter assembly in operable engagement with the actuation assembly; and
a trocar assembly in operable engagement with the adapter assembly, the trocar assembly including,
a housing including a tubular body having a distal portion and an end cap assembly secured to the distal portion;
a trocar member slidably supported within the housing between a retracted position and an advanced position, the trocar member including a proximal portion and at least one tab disposed on the proximal portion; and
a drive member rotatably supported within the housing and in operable engagement with the trocar member to cause longitudinal movement of the trocar member relative to the housing between the advanced position and the retracted position, wherein the at least one tab is configured to engage the end cap assembly when the trocar member is in the advanced position to prevent further advancement of the trocar member.
